(19) United States
(12) Patent Application Publication
(10) Pub. No.: US 2019/0291101 A1
(43) Pub. Date: Sep. 26, 2019

(54) PCR TUBE HOLDER

(71) Applicant: GOURGEN AMBARTSOUMIAN, LAVAL (CA)

(72) Inventor: GOURGEN AMBARTSOUMIAN, LAVAL (CA)

(21) Appl. No.: 16/139,730

(22) Filed: Sep. 24, 2018

Related U.S. Application Data
(60) Provisional application No. 62/701,116, filed on Jul. 20, 2018.
(60) Provisional application No. 62/647,121, filed on Mar. 23, 2018.

(51) Int. Cl. B01L 3/00

(52) U.S. Cl. CPC B01L 3/5085; C12M 23/58

(57) ABSTRACT

A PCR tube holder comprises an elongated strip body having a first portion and a second portion. One or more holes are defined in the first portion of the elongated strip body, the at least one hole configured to receive a single PCR tube. A writable zone in the second portion of the elongated strip body, the writable zone configured to receive ink thereon. A material of the elongated strip body is adapted to retain the ink through a PCR cycle. The material of the elongated strip body has a minimum flexural rigidity such that the elongated strip is adapted to support a PCR tube without folding.

### CROSS-REFERENCE TO RELATED APPLICATION

The present application claims the priorities of U.S. Patent Application No. 62/647,121, filed on Mar. 23, 2018, and of U.S. Patent Application No. 62/701,116, filed on Jul. 20, 2018, both of which are incorporated herein by reference.

### TECHNICAL FIELD

The present disclosure relates to polymerase chain reaction (PCR), and more particularly to the labelling and manipulating of PCR tubes, for example as regrouped as strips.

### BACKGROUND OF THE ART

Molecular biology is a branch of biochemistry that focuses on the molecular basis for biological activity within a cell, including the interactions between DNA, RNA and proteins. As such, many scientists, whether they work directly in molecular biology or another field often use molecular biology techniques to get a more complete understanding of the system they are researching. To that end, DNA has become a key marker is assessing the effects of various agents, from drugs to surgical intervention. By assessing changes in the transcription of the genome as a whole, or of specific transcripts, the role of different treatments may be better assessed.

PCR is a technique used in molecular biology to amplify a segment of DNA, across several orders of magnitude. PCR reactions have multiple possible applications. The technique can be used to assess the presence or absence of specific DNA fragments. This can be useful for genetic testing, forensic applications as well as research applications. PCR can also be used to amplify a target for selective DNA isolation or can be used to quantify precise changes in specific genes. This can be useful for genetic testing, cloning of genes, DNA sequencing, determining ethnicity and genealogy, forensic applications, identification and characterization of infectious agents, genetic fingerprinting, detection of mutations, as well as research applications.

During every cycle of a PCR reaction, primers will bind a target DNA sequence and in combination with a DNA polymerase and nucleotides will make a copy of the DNA sequence. As such, after every cycle, the number of that particular DNA sequence will have doubled. To accomplish this, the most common tool used is a PCR cycler, which can be programmed to cycle through the different temperatures required during the amplification process. The first step involves the denaturation of the DNA, to allow the primers to bind the desired sequence (˜95° C.). Following this there is the annealing/extension phase, during which the primers bind the DNA and a copy is produced (50° C.-65° C., 72° C.). Following this, the cycler will return to 95° C., and the cycle will begin anew, commonly for 30 to 40 times. The amplification cycles, protocols and conditions may have significant variations. The reaction itself is carried out in small PCR tubes, or an 8-tube PCR strips or PCR plates. PCR plates are known to have more than one row of tubes attached to each other, in contrast to the PCR strips that have a single row. These tubes/strips or plates are designed specifically to work in PCR thermo cycler and are able to withstand the variations in temperature. There are many variations of the PCR technology such as Real-Time PCR, Multiplex PCR, Nested PCR, RT-PCR, Quantitative PCR, Arbitrary Primed PCR. The PCR tubes, strips and plates in some occasions can be used for other type of laboratory sample preparation, manipulation and storage beyond their primary intended use for PCR.

Referring to FIG. 1 of the prior art, a PCR tube strip is generally shown at 10. The PCR tube strip 10 has a number of PCR tubes 12, held together in a strip by tabs 13. The PCR tubes 12 has a conventional shape of a conical closed bottom 12A, a cylindrical top 12B and an open end 12C. A cap 12D may or may not be integrally formed with the tubes 12 (FIG. 9). The tubes 12 are then capped prior to the PCR cycle and remain capped during the PCR cycle. FIG. 1 gives a perspective of the size of the PCR tubes 12 relative to fingers. However, due to the small size of PCR tubes, whether as individual units 12 or as part of multi-tube PCR strips 10 as in FIG. 1, the manipulating and labelling of PCR tubes and strips may be challenging.

### SUMMARY

It is an aim of the present disclosure to provide a PCR tube holder that addresses issues related to the prior art.

In accordance with an embodiment of the present disclosure, there is provided a PCR tube holder comprising: an elongated strip body having at least a first portion and a second portion, at least one hole defined in the first portion of the elongated strip body, the at least one hole configured to receive a single PCR tube, and a writable zone in the second portion of the elongated strip body, the writable zone configured to receive ink thereon, wherein a material of the elongated strip body is adapted to retain the ink through a PCR cycle, and further wherein the material of the elongated strip body has a minimum flexural rigidity such that the elongated strip is adapted to support a PCR tube without folding.

In accordance with the present disclosure, there is provided a PCR tube support comprising: an elongated strip body having a row of holes each configured to receive a single PCR tube of a PCR strip, and a writable zone configured to receive ink thereon, a material of the strip body being selected so as to retain the ink through a PCR cycle.

### DESCRIPTION OF THE DRAWINGS

FIG. 1 is a perspective view of a strip of PCR tubes in accordance with the prior art;

FIG. 2 is a top view of a PCR tube holder in accordance with an embodiment of the present disclosure;

FIG. 3 is a top view of the PCR tube holder of FIG. 2, with an alternative hole configuration;

FIG. 4 is a top view of the PCR tube holder of FIG. 2, with an alternative hole configuration;

FIG. 5 is a top view of a PCR tube holder in accordance with another embodiment of the present disclosure;

FIG. 6 is a top view of a PCR tube holder in accordance with another embodiment of the present disclosure;

FIG. 7 is a perspective view of a roll of the PCR tube holders in accordance with another embodiment of the present disclosure;

FIG. 8 is a top view of a PCR tube holder in accordance with another embodiment of the present disclosure, for supporting two PCR tube strips or multiple separated PCR tubes;

FIG. 9 is a perspective of a PCR tube strip in the PCR tube holder of FIG. 2;

FIG. 10 is a side view of the PCR tube strip in the PCR tube holder, showing a rigidity of the PCR tube holder;

FIG. 11 is a perspective view of a pair of PCR tube strips in the PCR tube holder of FIG. 8, in a PCR cycler;

FIG. 12 is a top view of interconnected PCR tube holders with a single hole, in accordance with another embodiment of the present disclosure;

FIG. 13 is a perspective view of the interconnected PCR tube holders of FIG. 12, in a roll;

FIG. 14 is a top view of interconnected PCR tube holders with a single hole and foldable tab, in accordance with another embodiment of the present disclosure;

FIG. 15 is a perspective view of the interconnected PCR tube holders of FIG. 14, in a roll;

FIG. 16 is a perspective view of PCR tube holders with a single hole, in transverse support of a PCR tube strip;

FIG. 17 is a top view of the PCR tube holder of FIG. 2, with an electronic chip;

FIG. 18 is a perspective view of PCR tube holders on a roll, with visual markers for automated printing equipment;

FIG. 19 is a top view of the PCR tube holder of FIG. 2, with a foldable tab;

FIG. 20 is a top view of the PCR tube holder of FIG. 2, showing exemplary positions for transverse perforations between holes

FIG. 21 is a perspective view of the interconnected PCR tube holders as in FIG. 12, with a widthwise perforation line;

FIG. 22 is a perspective view of the interconnected PCR tube holders in a roll as in FIG. 13, with a widthwise perforation line;

FIG. 23 is a perspective view of the interconnected PCR tube holders as in FIG. 12, with widthwise perforation lines;

FIG. 24 is a perspective view of the interconnected PCR tube holders in a roll as in FIG. 13, with widthwise perforation lines;

FIG. 25 is a top view of the interconnected PCR tube holders with colour coding;

FIG. 26 is a top view of PCR tube holders with a retaining rupture film;

FIG. 27 is a schematic layered view of a construction of a PCR tube holder on a support liner;

FIG. 28 is a schematic layered view of the construction of FIG. 27, showing examples of separation between the PCR tube holder and the support liner; and

FIG. 29 are schematic views of rolls of the PCR tube holder of the present disclosure with notch and mark positions.

### DETAILED DESCRIPTION

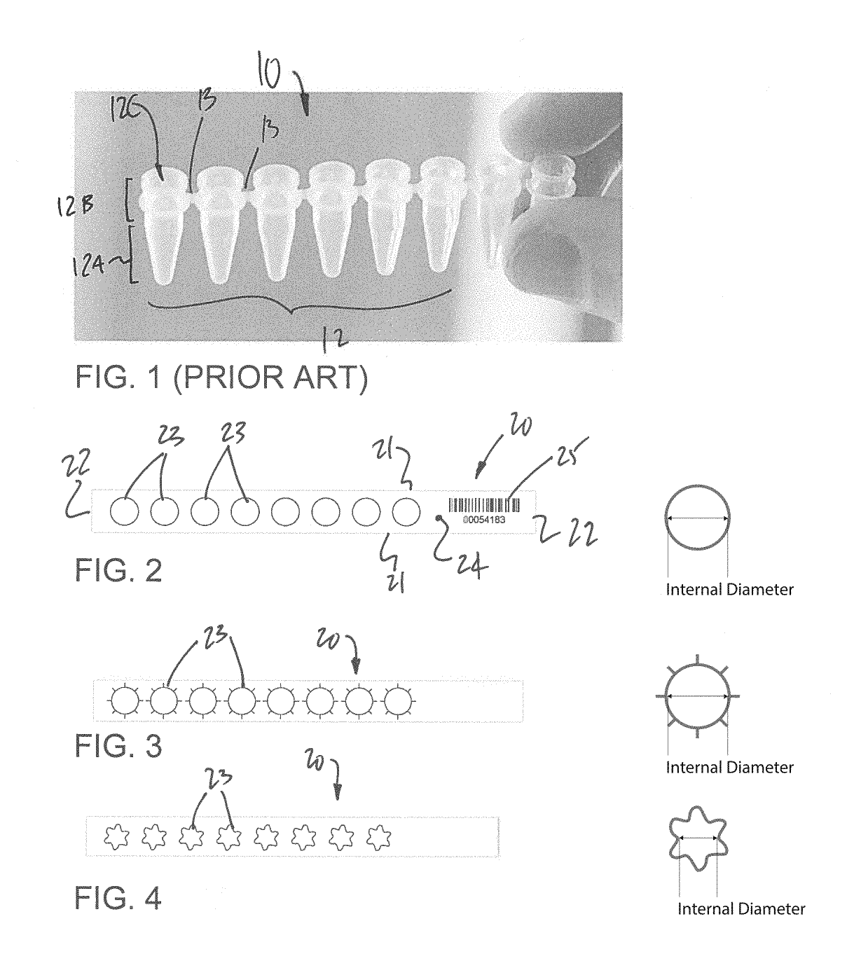

Referring to the drawings and more particularly to FIG. 2, a PCR tube holder in accordance with the present disclosure is generally shown at 20. The polymerase chain reaction (PCR) tube holder 20 is a strip of material, i.e., having a substantially smaller height than a length (the height may also be referred to as thickness, as it extends in the vertical direction when the holder 20 is used. The tube holder 20 may support information related to PCR strips, whereby it may also be known as a tag 20. The expression “tube holder 20” is used throughout the present disclosure, in spite of the fact that a PCR strip or PCR tube may be manipulated directly even in the presence of a tube holder 20, the tube holder 20 serving as a tag. In any event, the holder 20 “holds” the tubes, whereby the expression “holder” is appropriate when the holder 20 is used only as a tag. In a strip of material, the width may also be a fraction of the length of the strip. The PCR tube holder 20 is configured to support a PCR tube strip, such as the one shown at 10 in FIG. 1 of the prior art. While the expression PCR tube holder is used to describe item 20, other expressions may include tray, support, tag, identifier among other possible monikers. Moreover, while the present disclosure discusses the use of the PCR tube holder 20 with a strip 10 of PCR tubes 12, other types of tubes, single PCR tubes 12 (i.e. not attached to a strip), low profile and high profile PCR tubes 12, and PCR plates (a matrix of rows and columns, such as 3×8, 6×8, 12×8, etc) can benefit from the tube holder 20. This may include vials, other tubes, or PCR tubes 12 identified and manipulated with the tube holder 20 before, during and/or after PCR. As non-limitative examples of PCR tubes 12, tube strips and tube plates are Eppendorf™ 0.2 mL PCR Tube Strips (manufacturer part #951010022), Simport Scientific (manufacturer part # T320-2LPN, T320, T321 and T322 series, also series including T320-1, T320-2, T320-3, T322-1, T325-1, T325-2, T325-3, T325-4, T325-12, T323-24, T323-48, T323-96) all of which are incorporated herein by reference.

The PCR tube holder 20 has a strip body defined by a pair of elongated edges 21 extending along a longitudinal direction of the PCR tube holder 20. For instance, the elongated edges 21 are straight and parallel to the longitudinal direction, though they may have other peripheral outlines (e.g., wavy, with cutouts, etc). The strip body may further be defined by a pair of width edges 22 extending along a width direction of the PCR tube holder 20. For instance, the width edges 22 are straight and parallel to the width direction. Accordingly, in an embodiment, the strip body has a rectangular outline. Other shapes are considered, and tear-off holes may be present in the edges 21 and 22.

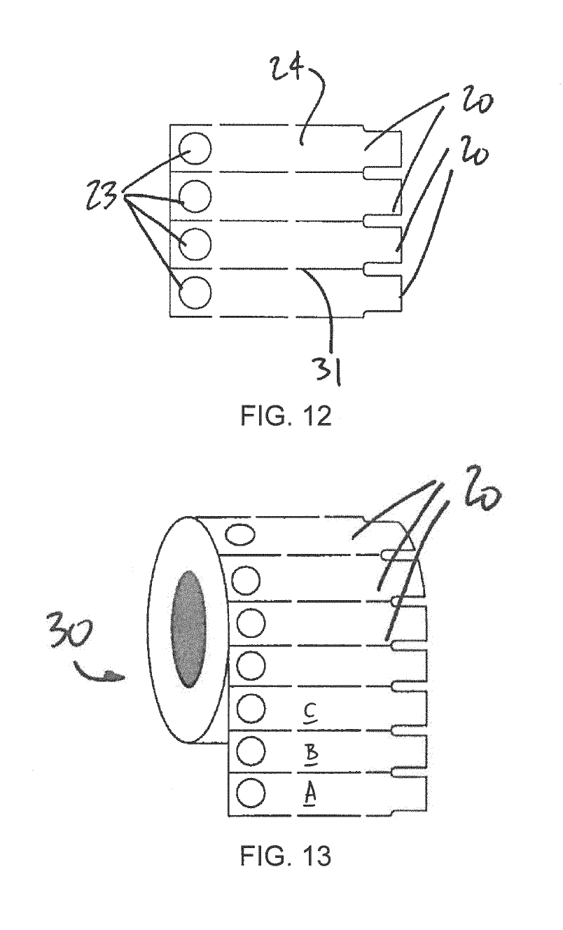

Holes 23 are formed through the strip body. In FIGS. 2 to 7, there are eight holes 23, to match the common eight tube configuration of the PCR strips 10. However, there may be as little as one hole 23 in the PCR tube holder 20. FIGS. 12-16 show embodiments of PCR tube holders 20 with a single hole 23, and are described hereinafter. According to an embodiment, the holes 23 are punched, die-cut, or laser-cut though the holes 23 may be formed in any other appropriate way. The holes 23 are shown as being circular, i.e., in conformity with the cross-sectional shape of the PCR tubes 12 along their vertical axes. The diameter of the holes 23 is therefore determined as a function of the portion of the outer diameter of the PCR tubes 12 upon which the PCR tube holder 20 will be attached. For example, in the PCR tubes 12 of FIG. 1, the diameter of the cylindrical top 12B is 6.35 mm±0.25 mm (other diameters are possible) (imperial, 0.25″±0.01″). Due to the presence of the tabs 13 between the tubes 12, the diameter of the holes 23 may be greater than 6.35 mm (imperial, 0.25″). However, it may also be less, to rely on deformation of the material of the PCR tube holder 20, which would result in a gripping action of the PCR tube holder 20 on the tubes 12. For example, a diameter of 6.00-6.25 mm for the holes 23 may provide a tighter grip (imperial, 0.236″-0.246″). According to an embodiment, the diameter, or smaller or wider diametrical dimension, ranges between 4.25 mm and 8.25 mm (imperial, 0.167″ and 0.325″). Furthermore, it is contemplated to have a PCR tube holder 20 with holes 23 having some difference in diameter within the same tube holder. For example, one or two holes 23 may have a tighter diameter for a stronger grip and the rest of the holes 23 would have bigger diameters for looser fit.

Also, the center-to-center spacing between the holes 23 is preferably equal to the center to center spacing of the PCR tubes 12 of a strip 10. Some variation may be present, which variation may rely on the stretchability or other deformation of the PCR tube holder 20 for the PCR tube holder 20 to be usable with the PCR strip 10. For example, as shown in 20, transverse perforation lines (i.e., perforations) or like creases may form fold lines to give some play against the variation.

Other shapes are considered, such as those shown in FIG. 3 and FIG. 4, being given as examples of numerous other shapes. In FIG. 3, the holes 23 are circular but radial slits are present and deformable to accommodate a PCR tube 12 that would have a diameter greater than that of the circular portion of the hole 23. In FIG. 4, the holes 23 are star-shaped, resulting in the presence of tabs that can also deform. When deformation is present, parts of the strip body that deform may act as tabs increasing a contact with the PCR tubes 12, to increase the grip between PCR tubes 12 and PCR tube holder 20. A combination of hole shapes within a same holder 20 is also possible.

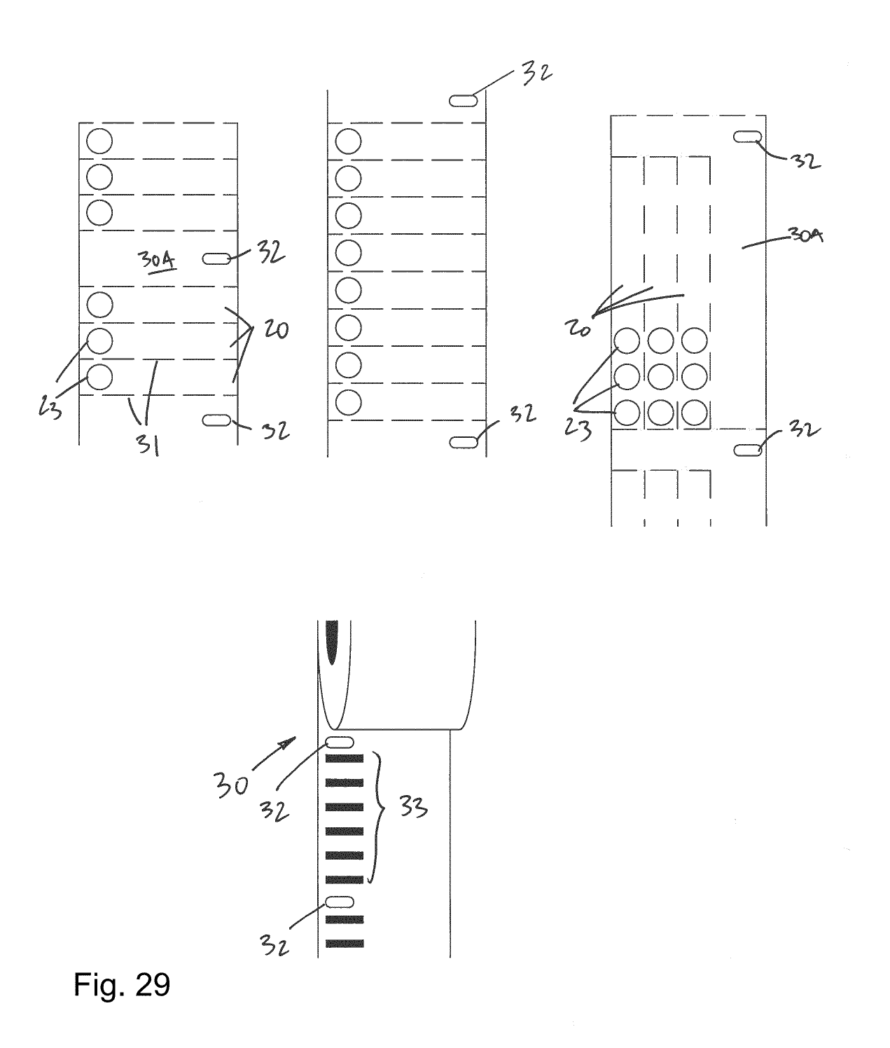

The PCR tube holder 20 may further include one of more writable field or zones 24. The writable zone 24 is devised to receive information or data thereon, in the form of ink from a marker, a pen, a pencil, a stamp, or from a printer, or originate from the direct thermal material from which the PCR tube holder 20 may be made. Other printing possibilities are considered, including ink jet, laser, thermal transfer, direct-thermal, thermal, flexographic, offset, digital, Liquid electrophotography, laser etching, impact printer, UV printer, type writer. Depending on the printer model requirements and configuration, the layout and the configuration of the PCR tube holder 20 can be adjusted or modified in order to be properly detected by sensor(s) and printed onto the printable area of the PCR tube holder 20. As a non-limitative example, a direct-thermal DYMO LabelWriter-450 or a similar model/printer might require notches 32 at a specific location above or below the PCR tube holders 20 in order to detect the position of the PCR tube holders 20, with FIG. 29 being provided as an example. This may entail the presence of non-PCR tube holder supports 30A spacing the PCR tube holders 20 to the appropriate printing positions. In some other models of the same printer, repetitive black (dark color) marks 33 or a combination of the marks and the notch(es) 32 may be present, and are shown as a non-limitative examples of configurations of the PCR tube holder 20 compatible with the DYMO printer. As another embodiment, sets of between 3 and 8 PCR tube holders 20 are configured per template for such a printer. Less than 3 and more than 8 PCR tube holders 20 per template are also contemplated. The templates can be positioned horizontally or vertically and comprise additional vertical perforations or other cuts, notches and marks depending on printer type, brand, model and software configuration. The data or indicia inscribed in the writable zone 24 may be in any appropriate form. For example, the data may be a barcode 25, with number below it, as an example of numerous possibilities including serial numbers, serial barcodes, variable or static data. Furthermore, the tube holder 20 may have a company logo or graphic image printed (e.g., pre-printed), or a date and/or time stamped, colour coding, etc. It is observed that, due to the elongated shape of the writable zone 24, the data extends in the longitudinal direction. As another embodiment, the writable zone 24 may incorporate or support a radio-frequency identification (RFID) chip or a near-field communication (NFC) chip 27 (as shown in FIG. 17 on a multi-hole holder, but adaptable to any configuration of the holders 20 described herein), as examples among others of electronic components. As an example, the zone 24 may be between 0.125″ and 2″ in the case of 8 hole holder 20 (metric, 3.18 mm and 50.8 mm). It may be longer if it can fit inside the PCR cycler. If the tube holder 20 comprises less than 8 holes. The writable zone 24 may be even longer.

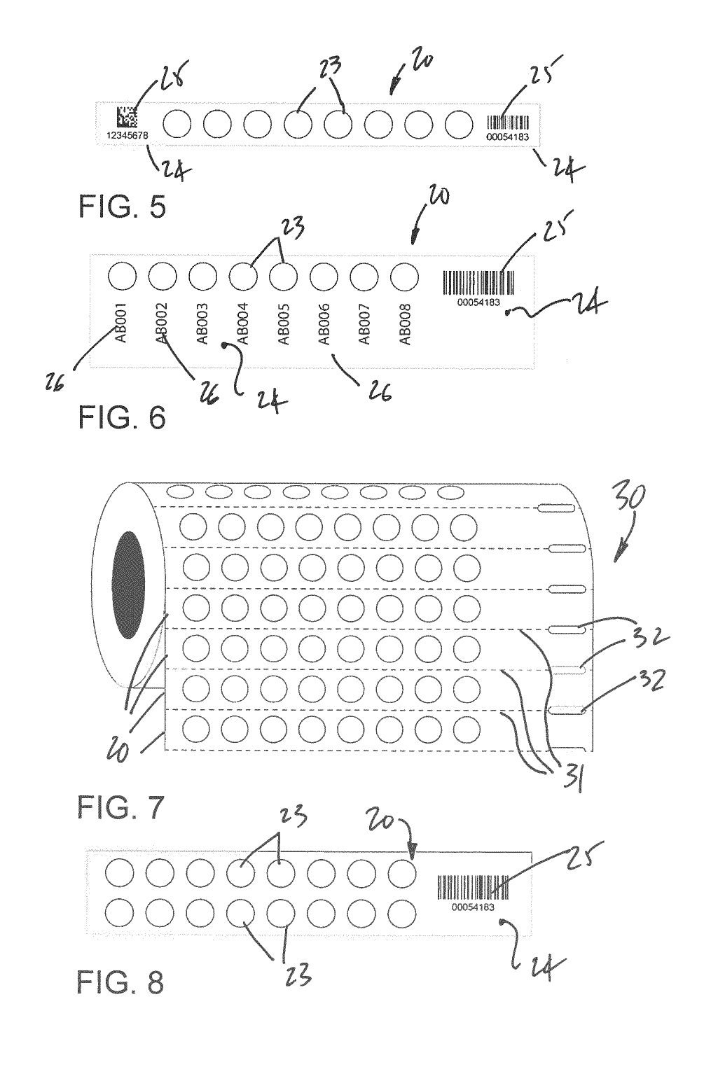

In FIGS. 2, 5 and 6, among others, the writable zone 24 may form a manipulation tab by which the PCR tube holder 20 may be manipulated, with the fingers spaced from the tubes 12 of the strip 10. Referring to FIG. 5, it is considered to shift the holes 23 to the right, to define a pair of writable zones 24 at opposite ends of the PCR tube holder 20. In such a case, the information may be printed in a different format or layout, and different type of information 25 may be provided at the opposite ends, as shown in FIG. 5. Moreover, the information 25 may be printed on both sides of the PCR tube holders 20, i.e., on the upper surface and/or on the undersurface of the strip body. A registration mark or an electronic chip may be provided on or incorporated in one or both surfaces of the tube holder 20 to be recognized by a printer, automation system or the robotic device in order to detect and print on the tube holder 20, manipulate the empty tube holder 20 or to manipulate the tube holder 20 loaded with a PCR tube 12, strip 10 or a PCR plate or to manipulate the tube holder 20 supporting a PCR tube, strip 10 or a PCR plate.

In the embodiment of FIGS. 2-5, the width edges 22 are larger than the diameter of the holes 23, with a minimum distance of 0.5 mm. It is possible to increase the width, to define additional writable zone surface, as in FIG. 6. In FIG. 6, the writable zone 24 extends in the width direction. In such a case, each of the holes 23 may be individually identified, in the manner shown in FIG. 6. Hence, the barcode 25 may be representative of the PCR strip 10, while each data item 26 may be associated to the PCR tube 12 that will be received in the respective hole 23. As also observed in FIG. 6, an orientation of the data items 26 may be aligned with the width direction, in such a way that the data items 26 are transverse relative to the data item 25. However, it is also contemplated to have all data items 25 and 26 aligned along the same direction.

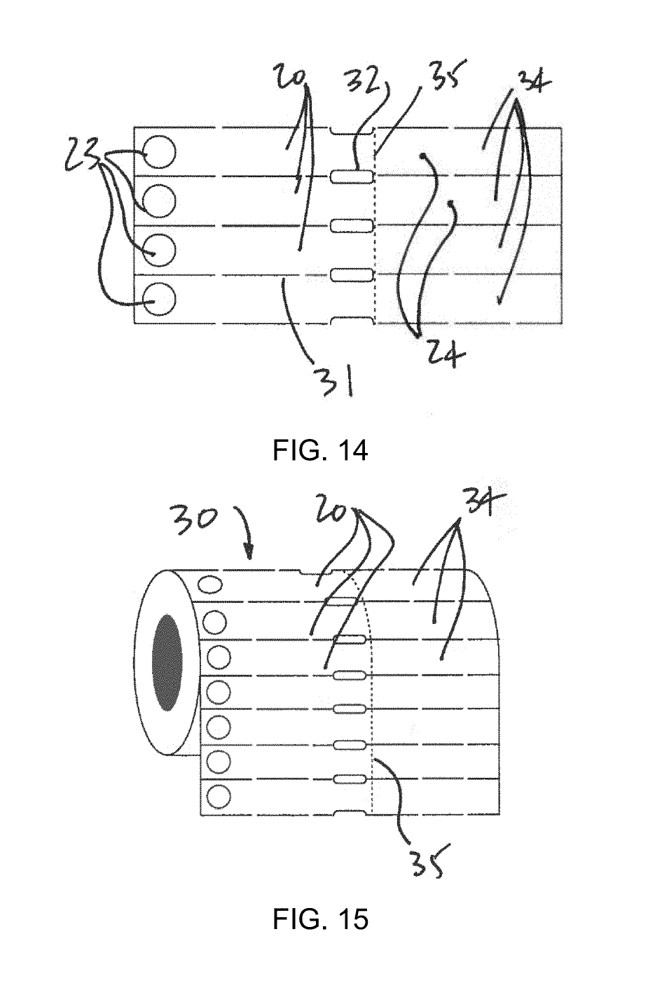

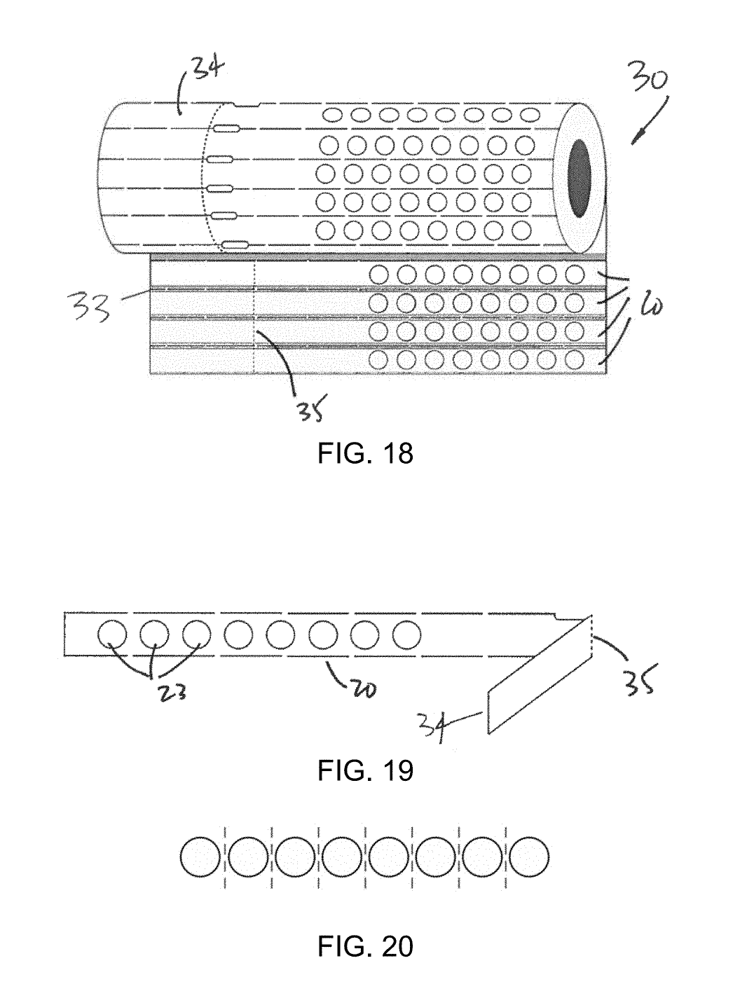

Referring to FIG. 7, the PCR tube holder 20 may be part of a roll 30. The roll 30 may be configured for being used with any appropriate commercial type roll printer, such as thermal-transfer, direct-thermal, inkjet, thermal, laser, impact. Spaced apart perforations may be present in the roll 30, to form tear strips 31, for tearing one of the PCR tube holder 20 away from the others in the roll. Tear-off holes or notches 32 may also be defined in the tear strips 31, to provide grip for a user tearing a PCR tube holder 20 away from the roll 30, and/or for being detected by optical sensors of printers. Although notches 32 and semi-notches may facilitate tearing off the PCR tube holders 20, such items 32 may be used for the determination of the tube holder position for printing in the appropriate location. Other elements may be incorporated to facilitate tearing (e.g. special cuts on the edges of perforation). The separation of the tube holders 20 can be achieved by combination of cuts and non-cut points. As shown in FIGS. 12-15 and 18, the edges of the roll 30 may start with a cut (i.e., transverse to 22). In an embodiment, at least one edge starts with a cut followed by a non-cut point of reinforcement (i.e., an attachment web, a non-perforated portion, points or strips of attachment etc). In another embodiment, both edges start with a cut followed by a non-cut point of reinforcement. The points of reinforcement are designed to maintain the integrity of the tube holders 20 during their production, printing and manipulating. Yet combination of the cuts/perforations and non-cut points of reinforcement assist in the tearing off the individual tube holders 20 and yet provide solid enough structure to avoid separation when pulled from the roll 30 during production, printing, and manipulation. The lengths of cuts/perforations and non-cut areas and their location can vary depending the material type, material caliper, material flexibility. The same tube holder 20 may have cuts of different lengths and reinforcement points of different lengths to provide an optimal tear experience and proper functionality of the system. As another embodiment the tags may have printed black marks or like dark marks for detecting the tube holders 20 and printing in appropriate location on the holder 20. The black marks or notches 32 can be used for the purpose of detection of the PCR tube holders 20 by optical sensors. It is contemplated to have both dark or black marks and notches 32 on the PCR tube holders 20. As another embodiment, instead of involving the manual tearing through a perforation(s), the sectioning of a single PCR holder 20 or a strip of a few PCR holders 20 can be done using a cutter accessory on a printer, such as by adding a cutting module on a CAB SQUIX printer or using a Zebra Z-class printer. In such a case, the knife of the cutter moves horizontally across the width of the PCR holder after the printing and slits it away. As shown in FIG. 18, a visual marker 33 may be provided to assist in the printing on the PCR tube holders 20, in particular for instances in which PCR tube holders 20 are interconnected to one another, such as in the roll 30, in a sheet, or as a fanfold, among possible embodiments. The visual markers 33 may be a line of contrasting color (e.g., a dark colour, UV reflective line, etc), a notch (e.g., as shown at 32), a gap for gap sensor, etc. The line can be through the entire length of the PCR tube holder 20 or on a portion of it only. Furthermore, marks for optical sensors may be of any shape such as a square, a rectangle or irregular shapes and combinations of multiple shapes. In some cases, the same PCR tube holder 20 or roll 30 may incorporate a combination of a notch and a lines or notches and a dark color shape(s) for detection by optical sensors. As shown in FIGS. 14, 15, and 17-19, the PCR tube holder 20 may also have a foldable tab 34. The foldable tab 34 may for instance be an integral part of the strip body, with a crease, a weakness, a fold, a visual mark, perforation lines, or any other like element 35, by which the foldable tab 34 may be folded toward a remainder of the strip body. This may be useful to reduce the length of the PCR tube holders 20 to fit in a PCR cycler, as an example, while not affecting the size of the writable zone 24. FIG. 19 shows the foldable tab 34 being folded. The foldable tab 34 may be on any of the tube holders 20 described herein. The foldable tab 34 also can be used to print a duplicate information for detaching and record keeping purposes, or for providing additional printable area for printing more information pertaining the content of the tube.

In the embodiments of FIGS. 2-7, the PCR tube holder 20 have one row of holes 23. It is however considered to have more than one row. For example, FIG. 8, shows a PCR tube holder 20 with two rows of holes 23. There may be more than two rows of holes 23 in a PCR tube holder 20.

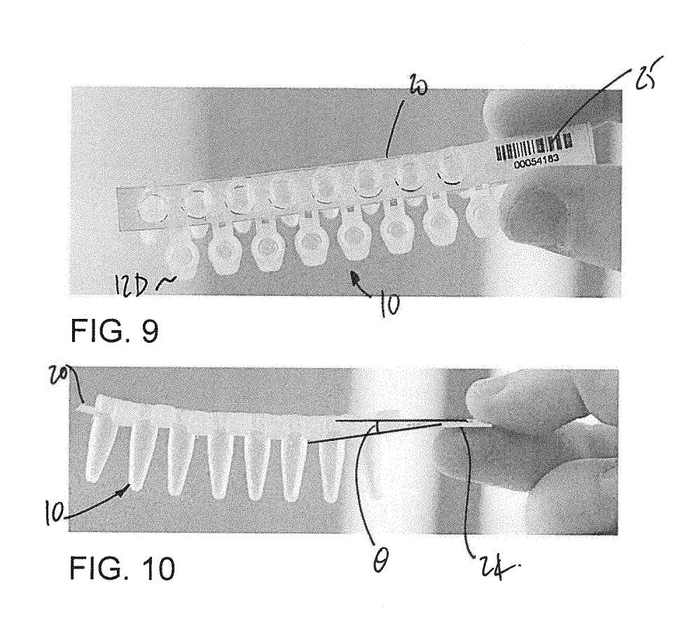

Referring to FIGS. 9 and 10, there is shown a PCR strip 10 in a PCR tube holder 20. As shown, each of the tubes 12 of the PCR strip 10 has its own dedicated hole 23. It is however contemplated to have less than the eight tubes 12 of the PCR strip 10 in the eight holes 23 of the PCR tube holder 20. It is further observed from FIG. 10 that the rigidity of the PCR tube holder 20 is such that the angle of deformation e (theta) of the portion of the PCR tube holder 20 supporting the strip 10 is around 15 degrees or less (or equal or less than 15 degrees) than relative to the manipulation tab integral to it, when supporting a filled PCR strip 10 (filled with 0.1 ml liquid and with closed caps 12D), or strips 10 in the case of the PCR tube holder 20 of FIG. 8, while held in cantilevered fashion with a pair of fingers as in FIG. 10 by an end. In another embodiment, the angle of deformation e (theta) of the portion of the PCR tube holder 20 supporting the strip 10 is about 30 degrees or less (or less equal or less than 30 degrees) relative to the manipulation tab integral to it. In yet another example, angle of deformation e (theta) of the portion of the PCR tube holder 20 supporting the strip 10 is about 45 degrees or less (or less equal or less than 45 degrees) relative to the manipulation tab integral to it. The rigidity may also be known and referred to as bending stiffness or flexural rigidity of the PCR tube holder 20. The PCR tube holder 20 has sufficient flexural rigidity so as not to fold when cantilevered by an end when supporting a PCR strip 10. In other words, the PCR tube holder 20 may bend as described above, but does not “break” to create a fold, i.e., it supports the PCR strip 10 without folding.

A suitable test method for determining the angle of deformation e (theta) of the portion of the PCR tube holder 20, and ensure it is less than 15 degrees relative to the manipulation tab integral to it, is set out as follows. An 8-hole PCR tube holder 20 as in FIGS. 2-7, having dimensions of length of 107 mm (4.2″), and of 9 mm of width (0.35″), is loaded with an 8-tube PCR strip 10 (e.g., T320-2R from Simport). Each tube of the PCR strip 10 contains 0.1 ml water. A clamp, such as a spring (e.g., 0.75″ wide) with a plastic sleeve, is used to grab the edge of the PCR tube holder 20, at 28.5 mm (approx. 1.125″) from the edge of the last hole 23, in the writable zone 24. The clamp is leveled horizontally and the bend in the PCR tube holder 20 is measured, using a protractor.

As discussed, herein, the PCR tube holder 20 may have fewer than eight holes 23. Accordingly, the test method may be adapted to shorter PCR tube holders 20. For example, a 3-hole PCR tube holder 20 having dimensions of length of 61 mm (2.4″), and of width of 9 mm (0.35″), is loaded with an 8-tube PCR strip 10 (e.g., T320-2R from Simport), each tube containing 0.1 ml water. Only three tubes are inserted inside the holes 23 of the PCR tube holder 20 and the remaining five tubes extend beyond the PCR tube holder 20. The clamp is used to grab the edge of the PCR tube holder 20, at 28.5 mm (approx. 1.125″) from the edge of the last hole 23, in the writable zone 24. The clamp is leveled horizontally and the bend in the PCR tube holder 20 is measured, using a protractor.

To reach this rigidity, the selection of material for the PCR tube holder 20 and its height are taken into consideration. According to an embodiment, plastics and polymers are used such as thermoplastic films. Polyesters (PET) may be used for their known rigidity, but other materials such as BOPP (biaxially oriented polypropylene), polypropylene, polyvinyl, or polyolefin, nylon, teslin are examples of numerous materials that may be used. Cardboard materials for example comprising fibres of cellulose also can be used for making tube holders 20. Use of composite materials or combination of materials is also contemplated. These materials can withstand or be treated to withstand PCR thermocycle conditions. In an embodiment, these materials may also be capable to withstand one or more of other laboratory conditions such as autoclaving and other sterilization methods, gamma irradiation, chemical exposures, cryogenic storage, and work equally well with frozen vials or cryogenically frozen vials.

According to an embodiment, a height of the PCR tube holder 20 for accommodating 1 strip or 1 tube is at least 0.25″ (6.35 mm). The height may range from 0.25″ to 0.5″ (6.35 mm-12.7 mm). The height can be larger if more information will be necessary to print on the tube holder to fit the parameters of the thermocycler unit. As an example, 7 mil-10 mil polyesters (PET) are well suited to be used as material for the PCR tube holder 20.

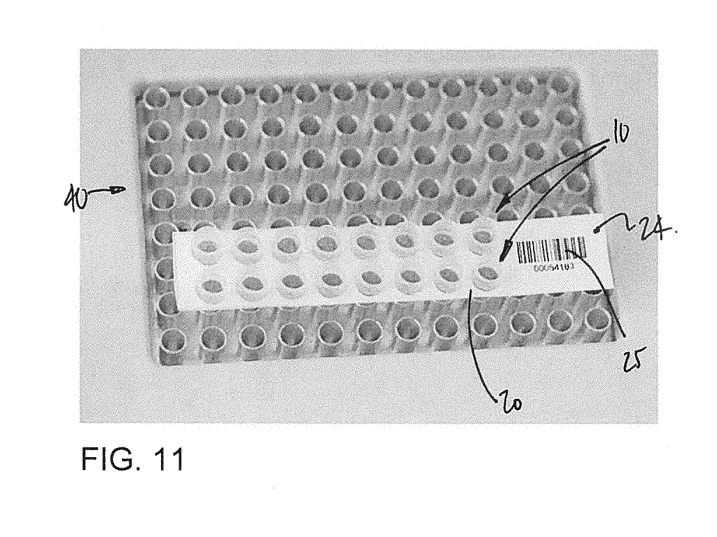

Referring to FIG. 11, the PCR strips 10 supported by the PCR tube holder 20 of FIG. 8 may be received in PCR cycler 40, with the PCR tube holder 20 remaining attached to the PCR strips 10, considering that the material of the PCR tube holder 20 is selected to withstand the conditions of PCR. The length of the PCR tube holder 20 may be determined to accommodate the size of the machine plate of the PCR cycler, which is commonly around 4.3″ (109 mm) wide and 3″ (77 mm long). The length of the PCR tube holder 20 may also be determined as a function of printer sizes. According to an embodiment, the PCR tube holders have a length ranging between 4.0″ (102 mm) and 8.5″ (216 mm) to fit standard barcode printers such as, non limitatively, a Zebra G-class or Z-class printer or a CAB Mach-1 printer or SQUIX printer. Yet as another embodiment the PCR tube holders have a length ranging between 0.375″ (9.5 mm) and 4.5″ (114 mm).

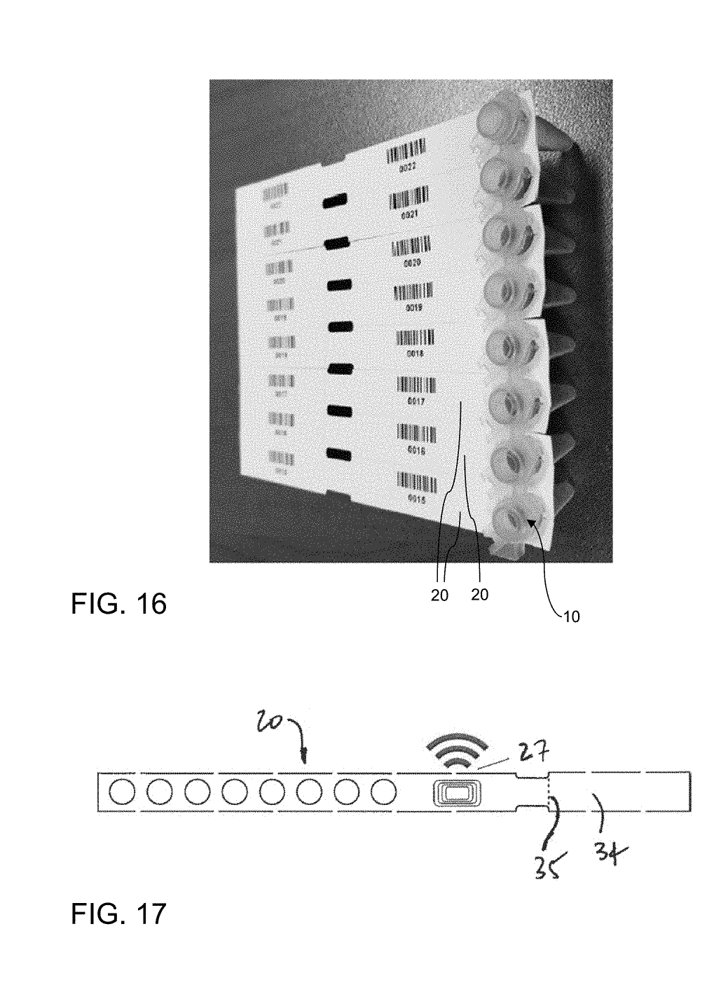

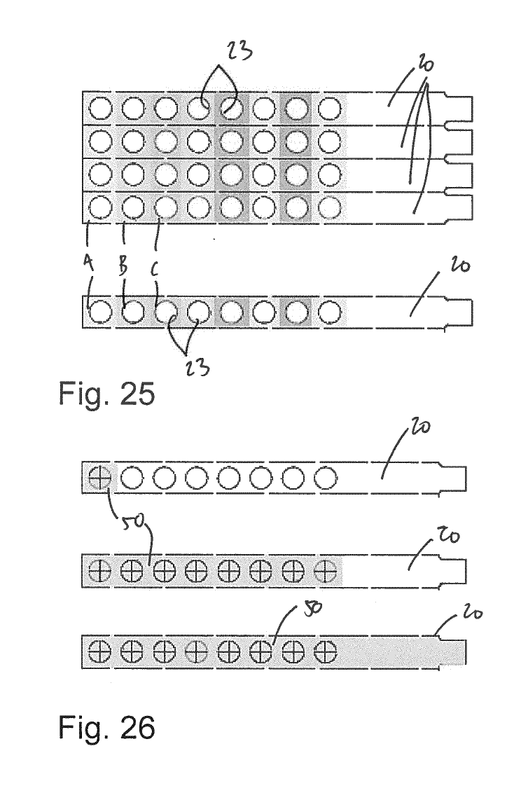

Referring to FIGS. 12-16, there are shown PCR tube holders 20 interconnected with one another, with the PCR tube holders 20 each having a single hole 23. Such PCR tube holders 20 may each be related to a respective PCR tube 12 of a PCR tube strip 10, with the potential for individual identification of each tube 12 by a dedicated tube holder 20, or even two or more tube holders 20 for each tube 12. Stated different, the configuration of FIGS. 12-16 is a single PCR tube holder 20 by PCR tube 12. This may allow rotation of the PCR tube holder 20 relative to the vertical axis of its PCR tube 12, for added flexibility. As observed from the arrangement of FIG. 16, the length of the PCR tube holders 20 may be oriented transversely and even perpendicular to the length of the PCR tube strip 10. In such a scenario, the PCR tube holders 20 may serve as a stand when the assembly of PCR tube strip 10 and PCR tube holders 20 is laid on a surface as in FIG. 16. Furthermore, the designs in FIG. 12-15 will allow to tear-off a cluster of more than one tube holder 20, such that the cluster may be used to tag multiple individual tubes or tube strips 10 or plates, for example as in FIG. 16: a cluster of eight PCR tube holders 20 can be teared away from the roll 30, the tube holders 20 remaining clustered, with the PCR tube strip 10 be inserted into the 8-hole cluster. If necessary the tubes 12 can be separated from each other and each tube 12 may remain connected to its individual tube holder 20. In another embodiment, an individual tube holder 20 may be applied to each end of PCR tube strip 10, thereby allowing identification of the strip 10 at both ends. It is also contemplated to use simultaneously more than one PCR tube holder 20 on for a same tube 12 or strip 10. As a non-limitative example, the PCR holder 20 shown in the FIG. 2-5 may be used on the same strip 10 as shorter PCR tube holders 20 as shown in FIG. 12-15, the shorter ones serving for example as identifier. This may allow individual identification of the tubes 12 of a stripe 10. It is also contemplated to provide a sequence of color to the interconnected PCR tube holders 20, for example as those of FIGS. 12-15. For example, as shown in FIG. 13, one tube holder 20 may be red A, the next may be blue B, the next may be yellow C (e.g., all colours light enough for printing thereon). The sequence may for instance repeat itself and may have a number of colours as a function of the number of tubes 12 in a strip 10, such as eight colours. Referring to FIG. 25, the PCR tube holder 20 may also have a sequence of colours to distinguish its holes 23. For example, each hole 23 has its own colour. It is also contemplated to group two or more adjacent or separated holes with a same colour. The colour may be used to give an identity to each of the PCR tubes 12 in the colour PCR tube holder 20 of FIG. 25, also known as colour-coding. The colour patterns shown in FIGS. 13 and 25 can be applied to PCR tube holders 20 with any number of holes 23. The use of colour codes may be used instead or in complement to printed data, such as data in the writable zone 24.

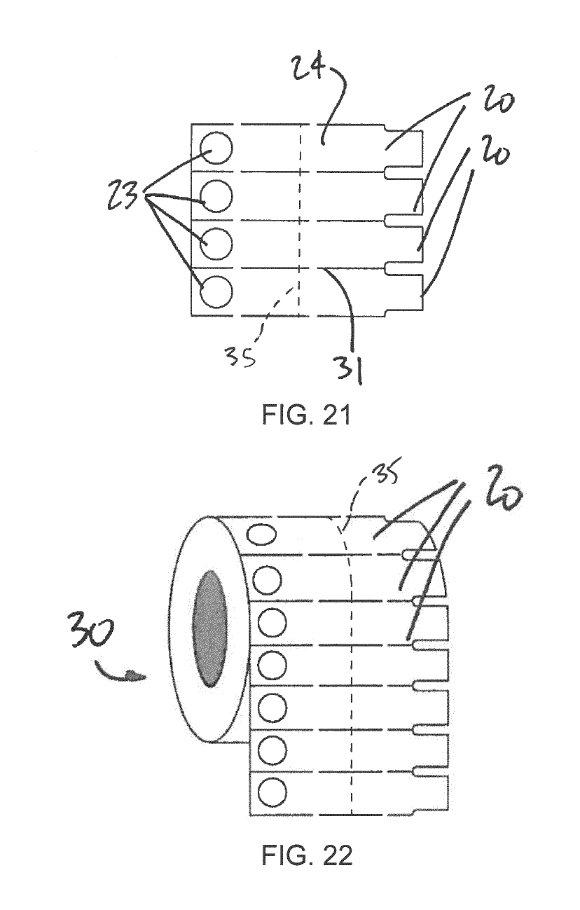

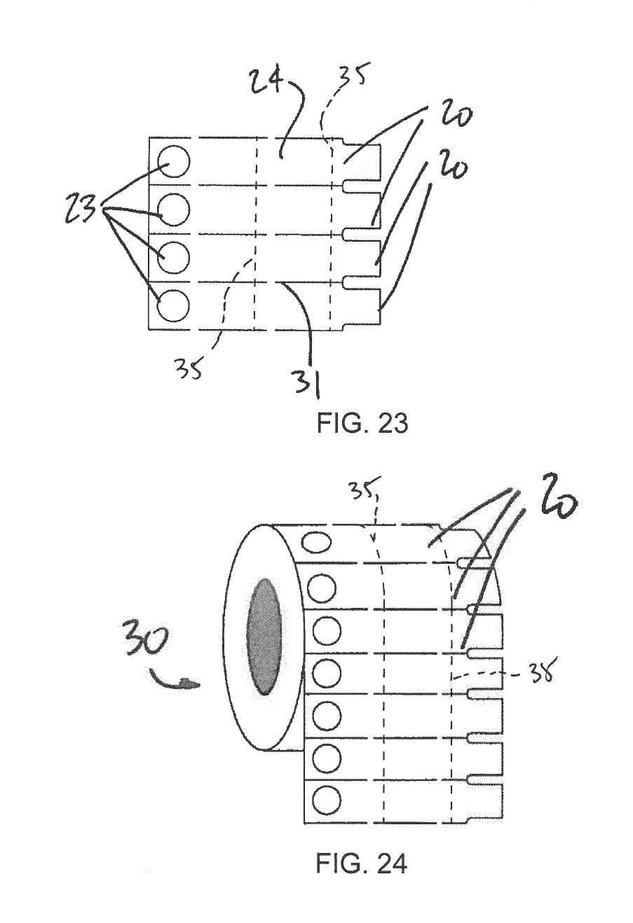

In another embodiment, the PCR tube holders 20 in FIGS. 12-15 may have perforation located after the notch or between the hole 23 and the notch 32 as shown in FIGS. 21 and 22, to allow a printer to print the PCR tube holders 20 using the notch 32 for proper positioning, with tear-away and removal of the end distal to the hole 23 through the widthwise perforation line 35. As in FIGS. 23 and 24, multiple widthwise perforation lines 35 be present, and may for example be parallel to one another. The perforation lines 35 can be used to select a desired size of the writable zone, with the rest being removable. In another embodiment, the information may be printed on two or more detachable sections, with one or more of the detached sections for placement in a notebook, for record keeping, or for other purposes.

As can be observed from the figures, the data 25 (e.g., barcode) is therefore horizontal, facing upwardly. As users typically look down on PCR strips, for instance when in a PCR cycler 40, the PCR tube holders 20 are in an ergonomic location, allowing for example cataloging from a bird's eye view which may facilitate proper identification of samples before, during and after PCR or their identification and retrieval during their prolonged storage for future use.

Referring to FIG. 26, another embodiment of the PCR tube holder 20 is shown, in which one or both of the surfaces of the PCR tube holder 20 are laminated with a retaining rupture film 50 with or without cuts or slits opposite the holes 23, shown as a cross in FIG. 26. For example, in FIG. 26, examples are shown of the film 50 being for one or more of the holes 23, and extending from end to end of the PCR tube holder 20 When forced through the film 50, a PCR tube ruptures and/or expands the thin film 50 and the PCR tube penetrates the hole 23. An adhesive may be layered in the film 50, whereby the rupturing of the film 50 exposes the adhesive that then surrounds the top part of the tube and helps in securing it in the hole 23. If no adhesive is present, the film 50 may nonetheless add friction to reinforce the connection between the periphery of the holes 23 and the tubes. In particular, the presence of the film 50 may be used with low-profile PCR tubes, that are mostly conical (i.e., no cylindrical portion or small cylindrical portion). The laminated retaining rupture film 50 can be made of a thermoplastic films such as a polyethylene, polyolefin, polypropylene, BOPP (biaxially Oriented Polypropylene), polyester, polyvinyl, polystyrene, nylon, acrylate, polyimide, etc.

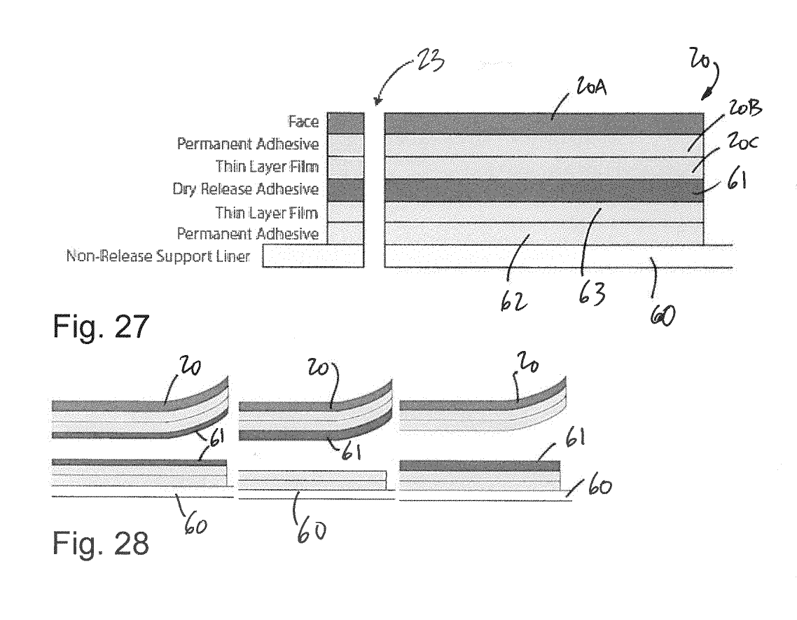

Referring to FIGS. 27 and 28, PCR tube holders 20 may be on a support liner 60 when grouped, for example to be printable as a sheet instead as in the roll 30. Referring to FIG. 27, an embodiment of a contemplated construction of the PCR tube holders 20 on the support liner 60 is shown. The PCR tube holder 20 may have a face, face layer or facestock 20A. The face 20A is the main component of the PCR tube holder 20 as it provides it with the structural integrity allowing the PCR holder 20 to have the rigidity described above. The face 20A may therefore consist of plastics, thermoplastics and polymers, such as PET, BOPP, polypropylene, polyvinyl, or polyolefin, polystyrene, nylon, acrylate, polyimide or cardboard materials comprising fibres of cellulose. In order not to have any tackiness when used, but to releasably adhere to the support liner 60, the face 20A may be connected to the support liner 60 by way of a dry release adhesive 61 layered between the face 20A and the support liner 60, that is not tacky when exposed in the manner shown in FIG. 28. To support the dry release adhesive 61, the face 20A may have a thin film layer 20C to which it is adhered via a permanent adhesive layer 20B. The adhesive layer 20B is qualified as being permanent in that the face 20A cannot be easily delaminated or separated from the thin film layer 20C. When the face 20A is pulley away from the support liner 60, separation will occur at the dry release adhesive layer 61, and not at the level of the adhesive layer 20B. The support liner 60 may have a similar construction, with to support the dry release adhesive 61, for instance by having its own thin film layer 63 to which it is adhered via a permanent adhesive layer 62.

In FIG. 27, the hole(s) 23 is shown as being punched through the assembly of the PCR tube holder 20 and support liner 60. However, the hole(s) 23 may be punched only in some of the shown layers, provided the layers constituting the PCR tube holder 20 are punched. As shown in FIG. 28, during separation of the PCR tube holder 20 from the support liner 60, the dry release adhesive 61 may remain on either one of the PCR tube holder 20 or support liner 60, or may be on both.

A kit may be provided as including one of more of the PCR tube holder 20, and one or more of a PCR tube 12, a PCR strip 10, a PCR plate, a printer, a scanner, a wireless communication device (e.g. RFID reader/encoder), a software or like application in machine readable code, an automation device or any combination thereof. In another embodiment, a PCR tube 12, PCR strip 10 or a PCR plate is provided with a PCR tube holder 20 pre-attached to it. In such a scenario, the PCR tube holder 20 may have some indicia or like data thereon, such as serial number, a barcode, alpha-numeric information, a graphic image, a logo or any combination thereof. In another embodiment, a computer template is provided for printing on the PCR tube holder 20 and/or instructions on how to format the PCR holder template for a computer or computerized use. In another embodiment, a computer program is provided for automation and robotic device to format template, print, scan or manipulate the PCR tube holder 20. PCR tube holders 20 may also be used as a booklet-type label and/or have more than one of the PCR tube holder 20 for each tube 12, for example with PCR tubes 20 of a same size (e.g., eight hole) or of different size (e.g., eight hole and three hole). The PCR tube holders 20 described herein may also have an overprint coating and/or additional layers of coatings or varnishes and/or additional layer(s) of material(s) laminated on one or both surfaces, to protect ink thereon or to attribute additional characteristics to the material of the PCR tube holder 20 or to add additional capabilities for any of its uses. The PCR tube holders 20 may be pre-cut individually, and be in a package such as in a plastic bag, box. The PCR tube holders 20 may be preprinted so that a lab technician can just pull out one and use it with tubes 12 ready for PCR.

Any of the PCR tube holders 20 or the applications thereof described herein can be used whenever applicable on an individual or single PCR tube 12 (not interconnected with each other) or PCR plates without any limitation.

1. A PCR tube holder comprising:
an elongated strip body having at least a first portion and a second portion,
at least one hole defined in the first portion of the elongated strip body, the at least one hole configured to receive a single PCR tube, and
a writable zone in the second portion of the elongated strip body, the writable zone configured to receive ink thereon,
wherein a material of the elongated strip body is adapted to retain the ink through a PCR cycle,
and further wherein the material of the elongated strip body has a minimum flexural rigidity such that the elongated strip is adapted to support a PCR tube without folding.

2. The PCR tube holder according to claim 1, further comprising at least one row of a plurality of the hole.

3. The PCR tube holder according to claim 2, wherein the row has three of the holes, a distance between an adjacent pair of the holes in the elongated strip body being equivalent to a distance between an adjacent pair of PCR tubes in a PCR strip.

4. The PCR tube holder according to claim 2, wherein the row has eight of the holes, a distance between an adjacent pair of the holes in the elongated strip body being equivalent to a distance between an adjacent pair of PCR tubes in a PCR strip.

5. The PCR tube holder according to claim 2, comprising two of the row, with each of the row having eight of the holes, a distance between an adjacent pair of the holes in any one of the rows of the elongated strip body being equivalent to a distance between an adjacent pair of PCR tubes in a PCR strip.

6. The PCR tube holder according to claim 2, wherein a diameter of the at least one hole is 6.35 mm±0.25 mm.

7. The PCR tube holder according to claim 1, wherein the material is one of thermoplastic film, PET, BOPP, polypropylene, polyvinyl, polyolefin, polystyrene, nylon, acrylate, polyimide or cardboard.

8. The PCR tube holder according to claim 1, further comprising a varnish on at least the writable zone.

9. The PCR tube holder according to claim 2, wherein the at least one row extends lengthwise in the elongated strip body, and wherein the writable zone is adjacent to the at least one row lengthwise.

10. The PCR tube holder according to claim 9, further comprising a third portion defining another writable zone configured to receive ink thereon.

11. The PCR tube holder according to claim 9, wherein the second portion and the third portion are on opposite ends of the first portion lengthwise.

12. The PCR tube holder according to claim 9, wherein the third portion extends widthwise relative to the first portion and to the second portion.

13. The PCR tube holder according to claim 2, further comprising pre-printed data in the writable zone.

14. The PCR tube holder according to claim 13, wherein the pre-printed data is a bar code.

15. The PCR tube holder according to claim 2, further comprising pre-printed data in the first portion for each of the holes, the pre-printed data being an identification of each of the holes.

16. The PCR tube holder according to claim 2, wherein a periphery of each said hole is pre-printed with a respective colour for colour coding.

17. The PCR tube holder according to claim 1, further comprising an electronic chip in the second portion.

18. The PCR tube holder according to claim 1, further comprising at least one tear-off perforation line extending widthwise.

19. The PCR tube holder according to claim 1, further comprising a rupture retaining film covering the hole.

20. The PCR tube holder according to claim 19, further comprising a slit in the rupture retaining film opposite the hole.

21. The PCR tube holder according to claim 2, wherein a diameter of the at least one hole is 6.00 mm±0.25 mm.

22. The PCR tube holder according to claim 2, wherein a diameter of the at least one hole is in the range of 4.25 mm to 8.25 mm inclusively.

23. A kit comprising:
at least one PCR tube holder as claims in claim 2; and
a strip of interconnected PCR tubes, with at least one of the PCR tubes received in one of the holes of the at least one row.
